(19) United States
(12) Patent Application Publication
(10) Pub. No.: US 2018/0235913 A1
(43) Pub. Date: Aug. 23, 2018

(54) AN ORAL PHARMACEUTICAL FORMULATION COMPRISING SUSTAINED-RELEASE GRANULES CONTAINING TAMSULOSIN HYDROCHLORIDE

(71) Applicant: HANMI PHARM. CO., LTD., Hwaseong-si Gyeonggi-do (KR)

(72) Inventors: Hyung Seo KIM, Hwaseong-si (KR); Young Keun CHOI, Suwon-si (KR); Jung Hyun CHO, Suwon-si (KR); Jin Cheul KIM, Seoul (KR); Yong Il KIM, Suwon-si (KR); Jae Hyun PARK, Suwon-si (KR); Jong Soo WOO, Suwon-si (KR)

(73) Assignee: HANMI PHARM. CO., LTD., Hwaseong-si, Gyeonggi-do (KR)

(21) Appl. No.: 15/551,194

(22) PCT Filed: Feb. 16, 2016
(86) PCT No.: PCT/KR2016/001538

§ 371 Date: Aug. 15, 2017

(30) Foreign Application Priority Data
Feb. 16, 2015 (KR) 10-2015-0023152

(51) Int. Cl. A61K 31/18; A61P 13/08; A61K 9/16

(52) U.S. Cl. CPC A61K 31/18; A61K 9/1635; A61K 9/1652; A61P 13/08

(57) ABSTRACT

An oral pharmaceutical formulation containing sustained-release granules including tamsulosin hydrochloride and a method of preparing the oral pharmaceutical formulation are provided. In the oral pharmaceutical formulation, the sustained-release granules includes about 10 parts to about 300 parts by weight of polyvinyl acetate, about 5 parts to about 250 parts by weight of hydroxypropyl methylcellulose, and about 1 part to about 450 parts by weight of a diluting agent with respect to 1 part by weight of tamsulosin hydrochloride, and a weight ratio of the sustained-release granules with respect to 1 part by weight of tamsulosin hydrochloride is about 360 to 495 parts by weight.

### TECHNICAL FIELD

The present disclosure relates to an oral pharmaceutical formulation containing sustained-release granules including tamsulosin hydrochloride, and more particularly, to an oral pharmaceutical formulation containing sustained-release granules including tamsulosin hydrochloride having stable efficacy and improved patient's compliance and a method of preparing the same.

### BACKGROUND ART

Tamsulosin hydrochloride, a α-adrenoceptor selective blocking agent, selectively acting on the urogenital organ is known to cause relaxation of smooth muscles surrounding the urinary bladder and prostate to thereby improve urination rate and benign prostatic hypertrophy symptoms with high drug efficacy and less side effects.

Tamsulosin hydrochloride has a relatively high bioavailability of 90% or greater, and has a half-life of about 9 to 13 hours in normal adults and a relatively long half-life of about 14 to 15 hours in benign prostatic hypertrophy patients. Accordingly, tamsulosin hydrochloride does not need to be prepared as a sustained-release formulation with prolonged release for about 12 to 24 hours or longer, and may maintain a sufficiently high concentration for 24 hours as a sustained-release formulation with prolonged release for only about 6 hours.

Tamsulosin hydrochloride as white crystalline powder is decomposable at a temperature of about 23 ° C., and has physiochemical characteristics with slight water solubility. Due to the water solubility of tamsulosin hydrochloride, the release rate of tamsulosin hydrochloride needs to be carefully controlled for sustained release.

Patent document 1 discloses a method of preparing a tamsulosin sustained-release formulation, the method including preparing granules including an enteric release controlling agent and filling capsules with the granules. However, such use of enteric release controlling agent may inhibit the constant release of an active ingredient in the intestinal fluid in which an enteric substance is dissolved, and may have the difficulty of representing constant release depending on pH changes or the presence of foods in the gastrointestinal track.

Patent document 2 discloses a capsule formulation containing tamsulosin hydrochloride sustained-release granules including tamsulosin hydrochloride, polyvinyl acetate, hydroxypropyl methylcellulose, and a granulating material. The capsule formulation uses pH-independent polyvinyl acetate and hydroxypropyl methylcellulose and thus it may ensure constant release of active ingredient, irrespective of pH change or the presence of foods in the gastrointestinal track. However, Patent document 2 only exemplifies a capsule formulation including about 150 mg or greater of the granules with respect to 0.2 mg of tamsulosin, i.e., about 750 parts by weight or greater of the final granules with respect to about 1 part by weight of tamsulosin hydrochloride. In other words, the capsule size of a single unit dosage form including a unit dose of tamsulosin may be too large for a patient to take it easily.

Currently commercially available tamsulosin hydrochloride capsules, including Flomax®, also include about 330 mg of granules with respect to 0.4 mg of tamsulosin hydrochloride, and consequentially have a large capsule size, and thus it may also not be easy for a patient to take them.

Furthermore, the capsule formulation of Patent document 2 and currently available tamsulosin hydrochloride capsule formulations have a small sphericity of granules and accordingly a large deviation in active ingredient dissolution rate.

### PRIOR ART DOCUMENT

### Patent Document

1. U.S. Pat. No. 4,772,475

2. WO 2005/077420 A1

### DETAILED DESCRIPTION OF THE INVENTION

### Technical Problem

The present disclosure provides an oral pharmaceutical formulation including sustained-release granules containing tamsulosin hydrochloride, having a reduced deviation in dissolution of the active ingredient due to a high sphericity of the granules, and improved patient's medication compliance with a reduced size of the final formulation.

The present disclosure provides a method of preparing the oral pharmaceutical formulation including the sustained-release granules containing tamsulosin hydrochloride.

### Technical Solution

According to an aspect of the present invention, there is provided an oral pharmaceutical formulation containing sustained-release granules including tamsulosin hydrochloride,

wherein the sustained-release granules includes about 10 parts to about 300 parts by weight of polyvinyl acetate, about 5 parts to about 250 parts by weight of hydroxypropyl methylcellulose, and about 1 part to about 450 parts by weight of a diluting agent with respect to 1 part by weight of tamsulosin hydrochloride, and a weight ratio of the sustained-release granules with respect to 1 part by weight of tamsulosin hydrochloride is about 360 to 495 parts by weight.

According to another aspect of the present invention, there is provided a method of preparing the above-described oral pharmaceutical formulation, the method including:

mixing and grinding a mixture of about 10 parts to about 300 parts by weight of polyvinyl acetate, about 5 parts to about 250 parts by weight of hydroxypropyl methylcellulose, and about 1 part to about 450 parts by weight of a diluting agent with respect to 1 part by weight of tamsulosin hydrochloride to form granules; and

spheronizing the formed granules with a spheronizer at a rotation speed of about 600 rpm to about 800 rpm for about 15 to about 35 minutes to obtain spheronized sustained-release granules.

### Advantageous Effects

According to the one or more embodiments of the present disclosure, an oral pharmaceutical formulation may have a smaller weight ratio of granules to active ingredient by about half, compared to conventional tamsulosin hydrochloride formulations, and thus a remarkably reduced size of the final single unit dosage form including the same unit dose of the active ingredient, compared to conventional formulations, and consequentially improved patient's medication compliance.

According to the one or more embodiments of the present disclosure, by the control of a weight ratio of the granules to the active ingredients and an amount ratio of polyvinyl acetate (PVAc) and hydroxypropyl methylcellulose (HPMC), the oral pharmaceutical formulation may have sustained release capacity of the active ingredient with a nearly zero order dissolution profile and reduced deviation in dissolution of the active ingredient due to an increased sphericity of the granules, compared to conventional formulations, and may consequentially exhibit stable efficacy. Therefore, an oral pharmaceutical formulation according to any of the embodiments may have pharmaceutical advantages including stable efficacy and, improved patient's compliance due to the patient's comfort with taking the oral pharmaceutical formulation.

### BRIEF DESCRIPTION OF THE DRAWINGS

FIG. 1 represents microscopic images of the granules separated from the capsule formulations of tamsulosin hydrochloride according to an example of the present disclosure (A in FIG. 1), a comparative example (B), and a control formulation (C, Flomax®, available from Boehringer Ingelheim);

FIG. 2 is a graph illustrating the results of a dissolution test on the capsule formulations of examples of the present disclosure and comparative examples prepared using different amounts of polyvinyl acetate, and the reference formulation (Flomax®, available from Boehringer Ingelheim), wherein the dissolution test was performed using a pH 1.2 aqueous buffer solution for 2 hours and subsequently using a pH 7.2 aqueous buffer solution for up to 8 hours;

FIG. 3 is a graph illustrating the results of a dissolution test on the capsule formulations of examples of the present disclosure and comparative examples prepared using different amounts of hydroxypropyl methylcellulose, and the reference formulation (Flomax®, available from Boehringer Ingelheim), wherein the dissolution test was performed using a pH 1.2 aqueous buffer solution for 2 hours and subsequently using a pH 7.2 aqueous buffer solution for up to 8 hours.

### Mode for Invention

Unless otherwise defined, all terms (including technical and scientific terms) used herein have the same meaning as commonly understood by one of ordinary skill in the art to which this invention belongs. Although exemplary methods or materials are listed herein, other similar or equivalent ones are also within the scope of the present invention. All publications disclosed as references herein are incorporated in their entirety by reference.

As a result of in-depth research into an oral pharmaceutical formulation including tamsulosin hydrochloride-containing sustained-release granules having a reduced deviation in dissolution of the active ingredient and improved patient's compliance due to a small size of the single unit dosage form, the inventors of the present application have completed the present invention based on the finding that by using polyvinyl acetate and hydroxypropyl methylcellulose in a specific weight ratio with respect to active ingredient to prepare sustained-release granules and controlling a weight ratio of the sustained-release granules to the active ingredient, the oral pharmaceutical formulation may have a reduced deviation in dissolution of the active ingredient due to a remarkably increased sphericity of the sustained-release granules, and a small size of a single unit dosage form due to a reduced weight ratio of the sustained-release granules to the active ingredient.

An aspect of the present disclosure provides an oral pharmaceutical formulation including sustained-release granules containing tamsulosin hydrochloride,

wherein the sustained-release granules includes about 10 parts to about 300 parts by weight of polyvinyl acetate, about 5 parts to about 250 parts by weight of hydroxypropyl methylcellulose, and about 1 part to about 450 parts by weight of a diluting agent with respect to 1 part by weight of tamsulosin hydrochloride, and

a weight ratio of the sustained-release granules with respect to 1 part by weight of tamsulosin hydrochloride is about 360 to 495 parts by weight.

The oral pharmaceutical formulation may be any solid formulation including the sustained-release granules without damage, for example, in the form of, but not limited to, granules, tablets, or capsules. For example, the oral pharmaceutical formulation may be in the form of a capsule including the sustained-release granules.

The oral pharmaceutical formulation, a weight ratio of the sustained-release granules of the oral pharmaceutical formulation with respect to 1 part by weight of tamsulosin hydrochloride 1 parts by weight may be about 360 parts to about 495 parts by weight, which is remarkably lower than a weight ratio of sustained-release granules to active ingredient compared to conventionally known capsule formulations of tamsulosin hydrochloride. Currently commercially available tamsulosin hydrochloride capsule formulations, including Flomax®, includes about 330 mg of granules per 0.4 mg of the active ingredient, i.e., about 800 parts by weight of the granules with respect to 1 part by weight of tamsulosin hydrochloride, and consequentially have a large size of the final capsule formulation, which may be too large to take it easily. Patent document 2 discloses a capsule formulation including about 300 mg or greater of sustained-release granules per 0.4 mg of active ingredient and thus having a large size of the final capsule formulation with respect to active ingredient. Accordingly, such conventional tamsulosin hydrochloride capsule formulations having a large single unit dosage form including 0.4 mg of a unit dose of the active ingredient may lead to low patient's compliance. In particular, in consideration of the fact that prostatic hypertrophy, an indication of tamsulosin hydrochloride is frequent in males over fifty and about 90% of the males over seventy suffers from prostatic hypertrophy, the size of tamsulosin hydrochloride capsule formulations is very important for patient's compliance.

The oral pharmaceutical formulation according to any embodiments may have a remarkably smaller size of a single unit dosage form including the same unit dose tamsulosin hydrochloride, compared to conventional formulations. In some embodiments, the oral pharmaceutical formulation as a capsule may use a hard capsule of any sizes that are generally used in medicine. Capsules are numbered differently depending on the sizes thereof and have different internal volumes. For example, a capsule of No. 00 has an internal volume of about 0.95 mL, a capsule of No. 0 has an internal volume of about 0.68 mL, a capsule of No. 1 has an internal volume of about 0.47 mL, a capsule of No. 2 has an internal volume of about 0.37 mL, a capsule of No. 3 has an internal volume of about 0.27 mL, and a capsule of No. 4 has an internal volume about 0.20 mL (refer to the website of Suheung Capsule Co., Ltd, Korea). An oral pharmaceutical formulation may use a small capsule for patient's compliance, but cannot use capsules of any sizes due to mass limit of the contents filled in a capsule. In some embodiments, the oral pharmaceutical formulation in the form of a capsule may use a capsule of No. 0, No. 1, No. 2, or No. 3, and in some embodiments, a capsule of No. 1, No. 2, or No. 3. For example, the oral pharmaceutical formulation may use a capsule of No. 3. In some embodiments, the oral pharmaceutical formulation in the form of a capsule may use a capsule of No. 3 that is enough to contain the sustained-release granules including 0.4 mg of tamsulosin hydrochloride. However, currently commercially available Flomax® formulation uses an elongated capsule of No. 2, longer than a capsule of No. 2. Therefore, the oral pharmaceutical formulation according to any embodiments may be convenient, in particular, for the elderly with reduced deglutition function to be taken with remarkably improved patient's compliance.

In some embodiments, the oral pharmaceutical formulation may have a sphericity of sustained-release granules of about 0.85 or greater, which is remarkably higher than that of conventional tamsulosin hydrochloride capsules including tamsulosin hydrochloride-containing sustained-release granules. This increased sphericity of the sustained-release granules in the oral pharmaceutical formulation is attributed to the fact that the sustained-release granules may include, with respect to 1 part by weight of tamsulosin hydrochloride, about 10 parts to about 300 parts by weight of polyvinyl acetate, about 5 parts to about 250 parts by weight of hydroxypropyl methylcellulose, wherein a weight ratio of the sustained-release granules with respect to 1 part by weight of tamsulosin hydrochloride is about 360 to 495 parts by weight. According to experimental results, the sustained-release granules of the oral pharmaceutical formulations had a significantly higher sphericity of about 0.85 or greater when the sustained-release granules includes about 10 parts to about 300 parts by weight of polyvinyl acetate and about 5 parts to about 250 parts by weight of hydroxypropyl methylcellulose and have a weight ratio of about 360 parts to about 495 parts by weight with respect to 1 part by weight of tamsulosin hydrochloride, compared to when the sustained-release granules are not within the above-described conditions (refer to Test Example 1). The oral pharmaceutical formulation according to any embodiments may exhibit stable efficacy of tamsulosin hydrochloride with reduced deviation in dissolution of tamsulosin hydrochloride due to the increased sphericity of the sustained-release granules (refer to Test Example 2).

In the oral pharmaceutical formulation according to any embodiments, the polyvinyl acetate (PVAc) may support a granulating material and forming pores in the granules for a period of time in an aqueous medium. Polyvinyl acetate may provide the oral pharmaceutical formulation with a consistent sustained release capability for an extended period of time in an aqueous medium regardless of pH level, and thus is an essential ingredient of the sustained-release granules. The polyvinyl acetate may have an average molecular weight of about 100,000 to about 500,000, but is not limited thereto.

In some embodiments, the polyvinyl acetate of the oral pharmaceutical formulation may be used alone or as a mixture with other materials, for example, in the form of powder or a diluted aqueous solution. For example, the polyvinyl acetate may be used as a mixture in powder form with any other water-soluble polymers such as polyvinylpyrrolidone. For example, Kollidon SR® (available from BASF), a powder prepared by spray-drying a 8:2 (w/w) mixture of polyvinyl acetate and polyvinylpyrrolidone, may be used.

In some embodiments, the polyvinyl acetate may be a diluted aqueous suspension of a mixture with other granulating materials, for example, Kollicoat SR30D® (available from BASF, solid content of about 30%) as a suspension of a mixture of polyvinyl acetate, polyvinylpyrrolidone, and sodium lauryl sulfate in water. Any material in any form including about 30% or more of polyvinyl acetate may be used as a source of the polyvinyl acetate. In some embodiments, the sustained-release granules may include about 10 parts to about 300 parts by weight, and in some embodiments, about 25 parts to about 150 parts by weight of polyvinyl acetate with respect to 1 part by weight of tamsulosin hydrochloride. When the amount of the polyvinyl acetate exceeds any of these ranges, the sustained release of the active ingredient may be too delayed. When the amount of the polyvinyl acetate is below any of these ranges, a quick release, not sustained, of the drug may occur (refer to Test Example 2).

In the oral pharmaceutical formulation according to any embodiments, the hydroxypropyl methylcellulose (HPMC) may control the dissolution rate of the active ingredient in the sustained-release granules. In particular, the hydroxypropyl methylcellulose may control the initial phase release of the active ingredient by forming pores as it is dissolved in an aqueous medium together with other ingredients, to thereby enable consistent sustained release of the active ingredient. The hydroxypropyl methylcellulose may have a viscosity of about 10,000 cPs or greater, and in some embodiments, of about 10,000 to about 100,000 cPs, and in some other embodiments, about 15,000 to about 100,000 cPs. Examples of hydroxypropyl methylcellulose having a viscosity within these ranges are METOLOSE 60SH, 65SH, and 90SH (available from Shin-Etsu Chemical Co., Ltd., Japan). When the viscosity of the hydroxypropyl methylcellulose is below any of these ranges, it may be difficult to achieve sustained release of the active ingredient in the sustained-release granules even with an increased amount thereof. For example, the amount of the hydroxypropyl methylcellulose in the sustained-release granules may be about 5 parts to about 250 parts by weight, and in some embodiments, about 5 to about 100 parts by weight with respect to 1 part by weight of tamsulosin hydrochloride. When the amount of the hydroxypropyl methylcellulose exceeds any of these ranges, the sustained release of the drug may be too delayed. When the amount of the hydroxypropyl methylcellulose is below any of these ranges, a quick release, not sustained, of the drug may occur (refer to Test Example 2).

The diluting agent may be a material able to maintain the volume of the granules constant. The diluting agent may be any diluting agents that are generally used in preparing granules. The diluting agent may be selected from microcrystalline cellulose, lactose, inorganic carriers such as dibasic calcium phosphate, dibasic calcium phosphate dihydrate, and tribasic calcium phosphate, and any combinations thereof, but is not limited thereto. For example, the amount of the diluting agent may be about 1 part to about 450 parts by weight, and in some embodiments, about 1 part to about 400 parts by weight, with respect to 1 part by weight of tamsulosin hydrochloride.

In some embodiments, the sustained-release granules of the oral pharmaceutical formulation may include about 25 parts to about 150 parts by weight of polyvinyl acetate, about 5 parts to about 100 parts by weight of hydroxypropyl methylcellulose, and about 1 part to about 400 parts by weight of the diluting agent, with respect to 1 part by weight of tamsulosin hydrochloride.

In some embodiments, the oral pharmaceutical formulation may have sustained release capability with a nearly zero order dissolution profile due to the inclusion of the sustained-release granules. For example, as evaluated using Dissolution method II (paddle method) in the U.S. Pharmacopoeia (USP) including a dissolution test in 500 mL of a pH 1.2 aqueous buffer solution at about 37±0.5° C. at about 100 rpm for about 2 hours, continuously followed by a dissolution test in a pH 7.2 aqueous buffer solution at about 37±0.5° C. at about 100 rpm for about 8 hours, the oral pharmaceutical formulation may have a dissolution rate of the tamsulosin hydrochloride of less than about 40 wt % as a result of the dissolution test for about 2 hours in the pH 1.2 aqueous buffer solution, and a dissolution rate of the tamsulosin hydrochloride of about 80 wt % or greater as a result of the dissolution test for about 8 hours in the pH 7.2 aqueous buffer solution.

In some embodiments, the sustained-release granules may be additionally coated with a sustained-release coating material. The sustained-release coating material may be an enteric coating material or polymeric coating material that are commonly used in the art. For example, the enteric coating material may be selected from hydroxypropyl methylcellulose phthalate, hydroxypropyl methylcellulose acetate succinate, polyvinyl acetate phthalate, cellulose acetate phthalate, shellac, a methacrylic acid-methyl methacrylate copolymer, a methacrylic acid-ethyl acrylate copolymer, and any combinations thereof, but is not limited thereto. For example, the polymeric coating material may be selected from hydroxypropyl methylcellulose, methylcellulose, ethylcellulose, polyvinyl acetate, and any combinations thereof, but is not limited thereto.

In some embodiments, the amount of the sustained-release coating material may be about 0.2 part to about 100 parts by weight, and in some other embodiments, about 1 part to about 50 parts by weight, with respect to 1 part by weight of tamsulosin hydrochloride.

In some embodiments, the sustained-release granules after granulation and an optional coating process may be formulated into any solid formulation without damage of the granules using a common method known in the art. For example, the sustained-release granules may be formulated as a capsule formulation.

In some embodiments, the oral pharmaceutical formulation may contain about 0.2 mg to about 0.8 mg of tamsulosin hydrochloride per single unit dosage form. In some embodiments, the oral pharmaceutical formulation may be a capsule formulation including the sustained-release granules. The capsule formulation may include about 0.4 mg of tamsulosin hydrochloride. The capsule formulation may contain the sustained-release granules including about 0.4 mg of tamsulosin hydrochloride in a capsule of No. 3. For information, currently commercially available or known prior-art capsule formulations of tamsulosin hydrochloride is a single unit dosage form containing a unit dose of about 0.4 mg of the active ingredient filled within a capsule of No. 2 or larger.

The oral pharmaceutical formulation according to any of the embodiments may be used for the treatment of any diseases which are known as an indication of tamsulosin hydrochloride, and for the treatment of any future diseases which will be identified as an indication of tamsulosin hydrochloride. As used herein, the expression “treatment” may include the meaning of “treatment”, “improvement”, “amelioration”, and “management” of a disease. In some embodiments, the oral pharmaceutical formulation according to any of the embodiments may be used for the treatment of, for example, benign prostatic hypertrophy, urination disturbances concomitant with prostatic hypertrophy, and acute urinary retention.

Another aspect of the present disclosure provides a method of preparing an oral pharmaceutical formulation of any of the embodiments, the method including:

mixing and grinding a mixture of about 10 parts to about 300 parts by weight of polyvinyl acetate, about 5 parts to about 250 parts by weight of hydroxypropyl methylcellulose, and about 1 part to about 450 parts by weight of a diluting agent with respect to 1 part by weight of tamsulosin hydrochloride to form granules; and

spheronizing the formed granules with a spheronizer at a rotation speed of about 600 rpm to about 800 rpm for about 15 to about 35 minutes to obtain spheronized sustained-release granules.

The above-detailed description of the oral pharmaceutical formulation according to any of the above-described embodiments may apply to description of the method of preparing the oral pharmaceutical formulation.

The forming of the granules may be performed using any granulation method known in the art. In some embodiments, the forming of the granules may be performed by wet-grinding the ingredients and extrusion granulating the wet ground products. For example, the extrusion granulating may be performed using an extruder into which the wet ground products are put.

The spheronizing of the formed granules may be performed using a spheronizer at a rotation speed of about 600 rpm to about 800 rpm for about 15 to about 35 minutes. When the rotation speed and the time are below these ranges, the resulting sustained-release granules may have a low sphericity. It may also be practically difficult to set the rotation speed above the range. When the spheronizing is performed for a time over the above range, the resulting sustained-release granules may have a reduced sphericity.

In some embodiments, the method of preparing an oral pharmaceutical formulation of any of the embodiments may further include filling a capsule with the spheronized sustained-release granules to form a capsule formulation. For example, a pharmaceutically acceptable additive may be optionally added to the spheronized sustained-release granules, if required, which may then be filled into a hard capsule to form a capsule formulation. For example, the pharmaceutically acceptable additive may be a plasticizer, a lubricating agent, or any other adjuvants.

### Mode of the Invention

One or more embodiments of the present disclosure will now be described in detail with reference to the following examples. However, these examples are only for illustrative purposes and are not intended to limit the scope of the one or more embodiments of the present disclosure.

### EXAMPLES 1-9 and COMPARATIVE EXAMPLES 1-8

### Preparation of Capsule Formulation Comprising Sustained-Release Granules Containing Tamsulosin Hydrochloride (1)

Tamsulosin hydrochloride, polyvinyl acetate (PVAc) (Kollicoat SR30D, available from BASF), hydroxypropyl methylcellulose (HPMC, METOLOSE 90SH), and a diluting agent were put into a high-speed mixer in a weight ratio as represented in Table 1, and an appropriate amount of water was added thereto, mixed together for about 10 minutes to about 15 minutes, and then wet-grinded. The resulting wet-grinded product was put into an extruder equipped with a sieve having a mesh size of about 0.8 mm and extruded at a screw speed of about 35 rpm, followed by spheronizing with a spheronizer at a rotation speed of about 750 rpm for about 26 minutes to obtain sustained-release granules of tamsulosin hydrochloride.

A coating solution including about 150.0 parts by weight of the sustained-release granules of tamsulosin hydrochloride, about 6.1 parts by weight (1.8 parts by weight as a solid ingredient) of polyvinyl acetate (PVAc, Kollicoat SR30D), about 0.4 parts by weight of povidone, about 0.3 parts by weight of propylene glycol as a plasticizer, and about 16.0 parts by weight of distilled water were sprayed using a NQ-160 fluidized bed system (available from DALTON, Japan) from the bottom thereof at an inlet temperature of about 35˜45° C., an outlet temperature of about 25˜35° C., a spray rate of about 7 to 13 RPM, and an spraying air pressure of about 500˜1000 m³/h to obtain the sustained-release granules of tamsulosin hydrochloride coated with the sustained-release coating material. The resulting sustained-release granules were filled into a hard capsule of No. 3.

TABLE 1
(Unit: mg/1 Capsule)

| | | | | Granulating | | |
|---|---|---|---|---|---|---|
| | | | | material | | |
| | Sustained-release coating | | Main ingredient | (diluting agent) | | |
| | material | | Tamsulosin | Microcrystalline | Coating | |
| | PVAc | HPMC | hydrochloride | cellulose | material | Total mass |
| Example 1 | 21.00 | 5.50 | 0.40 | 123.50 | 6.80 | 157.20 |
| Example 2 | 10.50 | 5.50 | | | | 146.70 |
| Example 3 | 31.50 | 5.50 | | | | 167.70 |
| Example 4 | 42.00 | 5.50 | | | | 178.20 |
| Example 5 | 52.50 | 5.50 | | | | 188.70 |
| Example 6 | 21.00 | 2.76 | | | | 154.46 |
| Example 7 | 21.00 | 11.00 | | | | 162.70 |
| Example 8 | 21.00 | 22.00 | | | | 173.70 |
| Example 9 | 21.00 | 33.00 | | | | 184.70 |
| Comparative | 0.00 | 5.50 | | | | 136.20 |
| Example 1 | | | | | | |
| Comparative | 6.00 | 5.50 | | | | 142.20 |
| Example 2 | | | | | | |
| Comparative | 63.00 | 5.50 | | | | 199.20 |
| Example 3 | | | | | | |
| Comparative | 84.00 | 5.50 | | | | 220.20 |
| Example 4 | | | | | | |
| Comparative | 21.00 | 0.00 | | | | 151.70 |
| Example 5 | | | | | | |
| Comparative | 21.00 | 0.40 | | | | 152.10 |
| Example 6 | | | | | | |
| Comparative | 21.00 | 44.00 | | | | 195.70 |
| Example 7 | | | | | | |
| Comparative | 21.00 | 66.00 | | | | 217.70 |
| Example 8 | | | | | | |

### EXAMPLE 10 and COMPARATIVE EXAMPLE 9

### Preparation of Capsule Formulation Comprising Sustained-Release Granules Containing Tamsulosin Hydrochloride (2)

Sustained-release granules of tamsulosin hydrochloride were prepared in the same manner as in Example 1, except that the rotation speed of the spheronizer was varied as represented in Table 2.

TABLE 2

| Example | Rotation speed (rpm) |
|---|---|
| Example 10 | 644 |
| Comparative Example 9 | 508 |

### EXAMPLES 11-12 and COMPARATIVE EXAMPLES 10-12

### Preparation of Capsule Formulation Comprising Sustained-Release Granules Containing Tamsulosin Hydrochloride (3)

Sustained-release granules of tamsulosin hydrochloride were prepared in the same manner as in Example 1, except that the rotation time of the spheronizer was varied as represented in Table 3.

TABLE 3

| Example | Rotation time (min) |
|---|---|
| Example 11 | 18 |
| Example 12 | 34 |
| Comparative Example 10 | 10 |
| Comparative Example 11 | 40 |

### Test Example 1

### Sphericity Test

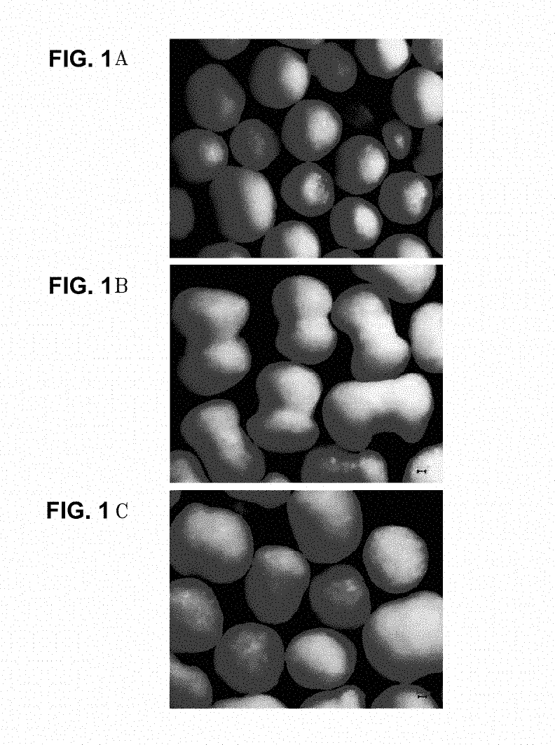

The sustained-release granules were separated from each of the capsule formulations of Examples 1 to 12 and Comparative Examples 1 to 6 as test formulations, and Flomax® capsules (available from Boehringer Ingelheim) as reference formulation to evaluate a sphericity of the sustained-release granules by microscopic observation of the surfaces of the sustained-release granules. The resulting microscopic images are shown in FIG. 1.

In particular, the evaluation of sphericity was performed as follows. First, a magnified view of a microscopic image of each granule was obtained using a microscope (Olympus BX51). After describing a circumscribed circle of each granule, the distance from the circumcenter to the surface of the granule was measure to obtain the maximum (“A”) and minimum (“B”) distances. The sphericity of each granule was evaluated by B/A. A granule having a value of B/A closer to “1” was determined as being closer to sphere. Ten granules from each formulation were used for the evaluation of sphericity, and an average and standard deviation of sphericities were calculated to thereby evaluate a sphericity of each formulation. The results are shown in Table 4.

TABLE 4

| | | | | | | | | | | | | Standard |
|---|---|---|---|---|---|---|---|---|---|---|---|---|
| Example | 1 | 2 | 3 | 4 | 5 | 6 | 7 | 8 | 9 | 10 | Average | deviation |
| Example 1 | 0.94 | 0.89 | 0.89 | 0.95 | 0.96 | 0.94 | 0.91 | 0.97 | 0.89 | 0.99 | 0.93 | 0.03 |
| Example 2 | 0.95 | 0.98 | 0.86 | 0.88 | 0.91 | 0.83 | 0.91 | 0.84 | 0.9 | 0.97 | 0.90 | 0.05 |
| Example 3 | 0.92 | 0.89 | 0.94 | 0.97 | 0.84 | 0.89 | 0.96 | 0.83 | 0.84 | 0.92 | 0.90 | 0.05 |
| Example 4 | 0.91 | 0.98 | 0.91 | 0.90 | 0.84 | 0.89 | 0.89 | 0.94 | 0.91 | 0.96 | 0.91 | 0.04 |
| Example 5 | 0.88 | 0.89 | 0.79 | 0.97 | 0.92 | 0.91 | 0.88 | 0.94 | 0.95 | 0.90 | 0.90 | 0.05 |
| Example 6 | 0.98 | 0.91 | 0.91 | 0.92 | 0.90 | 0.82 | 0.85 | 0.96 | 0.87 | 0.95 | 0.91 | 0.05 |
| Example 7 | 0.90 | 0.86 | 0.81 | 0.93 | 0.90 | 0.88 | 0.84 | 0.86 | 0.91 | 0.98 | 0.89 | 0.05 |
| Example 8 | 0.89 | 0.83 | 0.91 | 0.89 | 0.79 | 0.79 | 0.90 | 0.93 | 0.89 | 0.90 | 0.87 | 0.05 |
| Example 9 | 0.94 | 0.91 | 0.87 | 0.81 | 0.84 | 0.81 | 0.94 | 0.96 | 0.82 | 0.89 | 0.88 | 0.05 |
| Example 10 | 0.97 | 0.75 | 0.76 | 0.89 | 0.91 | 0.79 | 0.91 | 0.96 | 0.88 | 0.77 | 0.86 | 0.08 |
| Example 11 | 0.86 | 0.87 | 0.89 | 0.77 | 0.85 | 0.91 | 0.74 | 0.91 | 0.97 | 0.84 | 0.86 | 0.06 |
| Example 12 | 0.91 | 0.87 | 0.88 | 0.69 | 0.81 | 0.89 | 0.91 | 0.79 | 0.94 | 0.97 | 0.87 | 0.08 |
| Comparative | 0.24 | 0.56 | 0.55 | 0.46 | 0.49 | 0.4 | 0.31 | 0.70 | 0.31 | 0.42 | 0.44 | 0.13 |
| Example 1 | | | | | | | | | | | | |
| Comparative | 0.67 | 0.59 | 0.81 | 0.80 | 0.49 | 0.51 | 0.87 | 0.55 | 0.46 | 0.58 | 0.63 | 0.14 |
| Example 2 | | | | | | | | | | | | |
| Comparative | 0.87 | 0.83 | 0.74 | 0.79 | 0.86 | 0.78 | 0.81 | 0.96 | 0.69 | 0.91 | 0.82 | 0.08 |
| Example 3 | | | | | | | | | | | | |
| Comparative | 0.81 | 0.82 | 0.76 | 0.81 | 0.69 | 0.76 | 0.89 | 0.84 | 0.82 | 0.90 | 0.81 | 0.06 |
| Example 4 | | | | | | | | | | | | |
| Comparative | 0.42 | 0.59 | 0.28 | 0.71 | 0.59 | 0.46 | 0.21 | 0.68 | 0.51 | 0.49 | 0.49 | 0.15 |
| Example 5 | | | | | | | | | | | | |
| Comparative | 0.74 | 0.71 | 0.58 | 0.89 | 0.51 | 0.66 | 0.77 | 0.58 | 0.49 | 0.56 | 0.65 | 0.12 |
| Example 6 | | | | | | | | | | | | |
| Comparative | 0.59 | 0.81 | 0.89 | 0.51 | 0.47 | 0.55 | 0.54 | 0.69 | 0.81 | 0.73 | 0.66 | 0.14 |
| Example 7 | | | | | | | | | | | | |
| Comparative | 0.54 | 0.40 | 0.78 | 0.39 | 0.57 | 0.37 | 0.39 | 0.73 | 0.50 | 0.44 | 0.51 | 0.14 |
| Example 8 | | | | | | | | | | | | |
| Comparative | 0.64 | 0.59 | 0.82 | 0.71 | 0.63 | 0.58 | 0.64 | 0.71 | 0.78 | 0.81 | 0.69 | 0.08 |
| Example 9 | | | | | | | | | | | | |
| Comparative | 0.51 | 0.77 | 0.57 | 0.66 | 0.63 | 0.47 | 0.69 | 0.79 | 0.6 | 0.69 | 0.64 | 0.10 |
| Example 10 | | | | | | | | | | | | |
| Comparative | 0.88 | 0.75 | 0.59 | 0.85 | 0.9 | 0.68 | 0.77 | 0.83 | 0.91 | 0.73 | 0.79 | 0.10 |
| Example 11 | | | | | | | | | | | | |
| Reference | 0.72 | 0.89 | 0.91 | 0.87 | 0.69 | 0.78 | 0.8 | 0.83 | 0.68 | 0.87 | 0.80 | 0.08 |
| formulation | | | | | | | | | | | | |

Referring to Table 4, an average sphericity of the sustained-release granules of each of the capsule formulations of Examples 1 to 12 are remarkably closer to “1”, compared to those of the capsule formulations of Comparative Examples 1 to 11 and the reference formulation. Therefore, sustained-release granules according to embodiments (Examples 1 to 12) may have remarkably higher sphericity and be closer to a complete sphere, compared to those of Comparative Examples 1 to 11 and the reference formulation, and have a remarkably smaller standard deviation of sphericity, indicating that the sustained-release granules according to embodiments (Examples 1 to 12) may be more uniform in size and more homogeneous, compared to those of Comparative Examples 1 to 11 and the reference formulation.

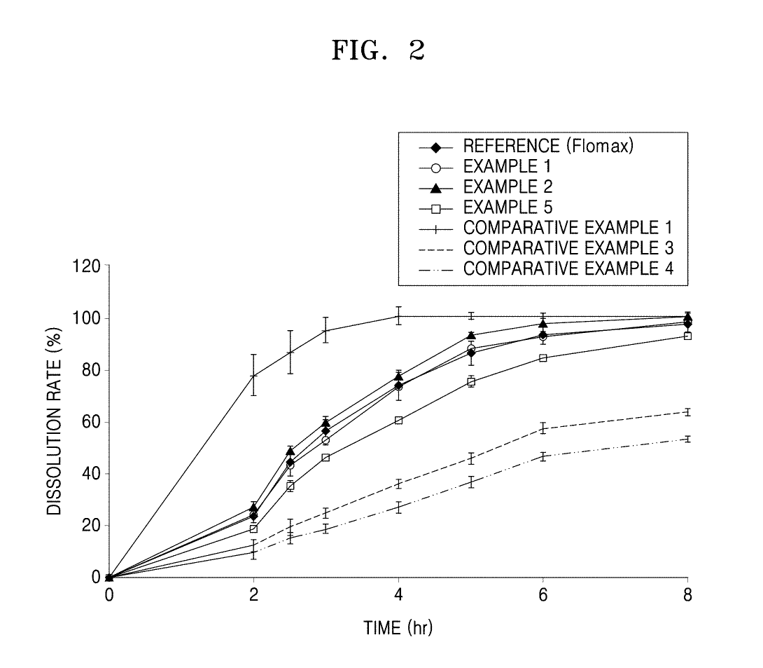

The sustained-release granules of Comparative Example 3 and Comparative Example 4 had a higher average sphericity, compared to the reference formulation, but had a too low dissolution rate (see Tables 5, 6 and 7 and FIG. 2 in Test Example 2)

### Test Example 2

### Dissolution Test

A dissolution test of tamsulosin hydrochloride (0.4 mg) was performed on the capsule formulations of Examples 1, 2, 5, 6, and 9 and Comparative Example 1, 3, 4, 5, and 8 as test formulations, and Flomax® capsule (available from Boehringer Ingelheim) as reference formulation. The dissolution test was begun with a strong acid artificial gastric juice as a dissolution medium, which was then changed after 2 hours with a neutral phosphate buffer solution. Detailed dissolution test conditions are as follows.

<Dissolution Test Conditions >

Dissolution medium: 1) 500 ml of artificial gastric juice (pH 1.2)+1 ml of Tween 80
2) 500 ml of phosphate buffer solution (pH 7.2)

Dissolution medium temperature: 37±0.5° C.

Paddle speed: 100 rpm

<Sampling and Analysis Method >

About 10 ml of the dissolution medium was taken after 2 hours, 2.5 hours, 3 hours, 4 hours, 5 hours, 6 hours, and 8 hours from the start of the dissolution test. After about 2.0 ml of an internal standard solution (a 0.0005% w/v solution of propylparaben in a mixture of water and acetonitrile (7:3)) was added to each of the sample solutions taken from the strong acid dissolution medium, and about 1.0 ml of 0.5N HCl solution and then about 2.0 ml of the internal standard solution were added to each of the sample solutions taken from the neutral dissolution medium, the sample solutions were filtered using a membrane filter having a pore size of about 0.45 μm or less. Each of the filtrates were analyzed by liquid chromatography (Column—Cosmosil C18 ODS (4.6×150 mm, 5 μm), Temperature—about 40° C., Mobile phase—a 7:3 mixture of aqueous perchloric acid solution (adjusted to about pH 2.0 with sodium hydroxide) and acetonitrile) at a flow rate of about 1.0 ml/min (Injection volume—about 500 μl) using a UV detector at about 225 nm.

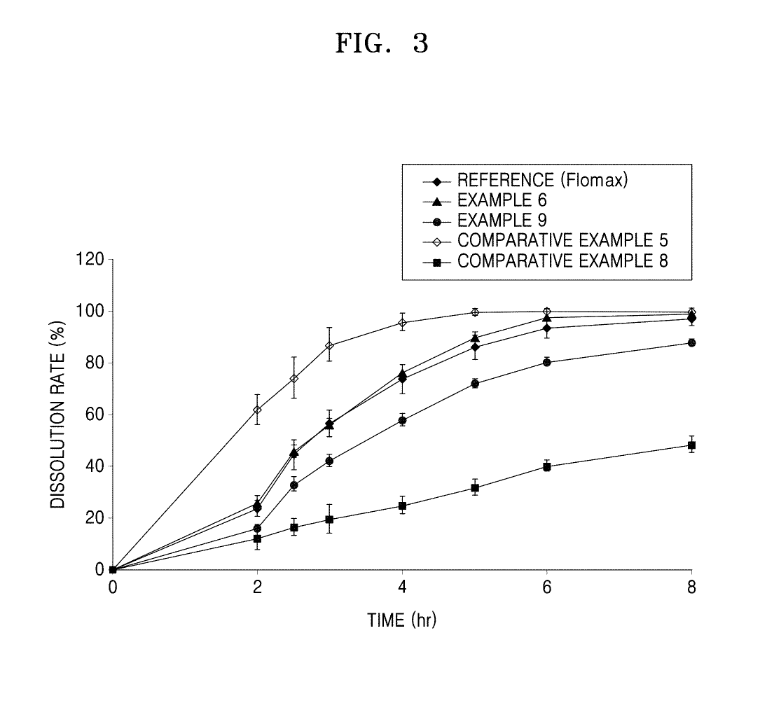

The resulting dissolution rates of tamsulosin hydrochloride in the capsule formulations and the reference formulation are shown in Table 5 and FIGS. 2 and 3. A standard deviation of the dissolution rates in each formulation are shown in Table 6, and an average standard deviation of the dissolution rates in each formulation are shown in Table 7.

TABLE 5
Dissolution rate

| | Reference | | | | | | | | | | |
|---|---|---|---|---|---|---|---|---|---|---|---|
| Time | formulation | Example | | | | | Comparative Example | | | | |
| (hr) | (Flomax ®) | 1 | 2 | 5 | 6 | 9 | 1 | 3 | 4 | 5 | 8 |
| 0 | 0.0 | 0.0 | 0.0 | 0.0 | 0.0 | 0.0 | 0.0 | 0.0 | 0.0 | 0.0 | 0.0 |
| 2 | 23.7 | 24.5 | 27.1 | 18.8 | 26.2 | 16.3 | 77.8 | 12.3 | 9.8 | 62.0 | 12.1 |
| 2.5 | 44.5 | 43.3 | 48.7 | 35.2 | 45.8 | 33.1 | 86.5 | 19.3 | 15.1 | 74.5 | 16.7 |
| 3 | 56.4 | 52.9 | 59.6 | 46.1 | 56.6 | 42.3 | 95.0 | 24.9 | 18.8 | 87.1 | 19.7 |
| 4 | 73.8 | 73.1 | 77.4 | 60.5 | 76.8 | 58.1 | 100.6 | 35.9 | 27.0 | 95.8 | 25.0 |
| 5 | 86.1 | 88.5 | 93.1 | 75.4 | 90.3 | 72.2 | 100.6 | 45.7 | 36.8 | 99.8 | 32.1 |
| 6 | 93.5 | 92.8 | 98.1 | 84.5 | 98.2 | 80.7 | 100.4 | 57.3 | 46.5 | 100.1 | 40.4 |
| 8 | 97.4 | 98.9 | 100.8 | 92.9 | 99.5 | 88.1 | 100.8 | 63.6 | 53.1 | 99.9 | 48.7 |

TABLE 6
Standard deviation of dissolution rates

| | Reference | | | | | | | | | | |
|---|---|---|---|---|---|---|---|---|---|---|---|
| Time | formulation | Example | | | | | Comparative Example | | | | |
| (hr) | (Flomax ®) | 1 | 2 | 5 | 6 | 9 | 1 | 3 | 4 | 5 | 8 |
| 0 | 0.00 | 0.00 | 0.00 | 0.00 | 0.00 | 0.00 | 0.00 | 0.00 | 0.00 | 0.00 | 0.00 |
| 2 | 3.02 | 1.57 | 2.01 | 1.34 | 2.06 | 1.06 | 7.95 | 2.21 | 2.84 | 5.87 | 4.15 |
| 2.5 | 5.69 | 0.58 | 1.51 | 2.31 | 2.48 | 2.66 | 8.12 | 3.17 | 2.01 | 7.91 | 3.10 |
| 3 | 5.21 | 1.56 | 0.93 | 1.38 | 1.79 | 2.29 | 5.03 | 1.92 | 1.74 | 6.56 | 5.66 |
| 4 | 5.75 | 1.31 | 1.33 | 1.35 | 2.10 | 2.45 | 3.41 | 1.88 | 2.10 | 3.41 | 3.41 |
| 5 | 4.73 | 2.15 | 0.76 | 2.11 | 1.77 | 1.88 | 1.31 | 2.11 | 2.31 | 1.44 | 3.11 |
| 6 | 3.95 | 1.63 | 1.21 | 1.02 | 0.96 | 1.48 | 0.98 | 1.99 | 1.52 | 0.98 | 2.09 |
| 8 | 3.11 | 0.74 | 0.63 | 0.97 | 0.97 | 1.40 | 1.03 | 1.31 | 1.09 | 1.23 | 3.23 |

TABLE 7
Average standard deviation of dissolution rates after 2-4 hours

| | Reference | | | | | | | | | | |
|---|---|---|---|---|---|---|---|---|---|---|---|
| | formulation | Example | | | | | Comparative Example | | | | |
| | (Flomax ®) | 1 | 2 | 5 | 6 | 9 | 1 | 3 | 4 | 5 | 8 |
| Average | 4.92 | 1.25 | 1.45 | 1.60 | 2.11 | 2.12 | 6.13 | 2.30 | 2.17 | 5.94 | 4.08 |
| standard | | | | | | | | | | | |
| deviation | | | | | | | | | | | |

According to the dissolution test results, the capsule formulations of Example 1, 2, 5, 6, and 9 had a similar zero order dissolution profile to the reference formulation. Due to high sphericities, the capsule formulations of Examples 1, 2, 5, 6, and 9 had an average standard deviation in dissolution rate of about 2 or less after 2 to 4 hours from the dissolution test (i.e., before the dissolution rate reaches 100%), which was significantly lower than those of the reference formulation and the comparative Examples.

Therefore, a pharmaceutical formulation according to an embodiment may maintain dissolution rate constant between products from a single production batch or between products from different production batches, and consequentially may exhibit consistent drug efficacy.

The capsule formulations of Comparative Examples 3 and 4 appear to have a small standard deviation in dissolution rate, but may not have a satisfactory drug efficacy due to a very low dissolution rate thereof resulting from the inclusion of excess polymer.

While this invention has been particularly shown and described with reference to preferred embodiments thereof, it will be understood by those skilled in the art that various changes in form and details may be made therein without departing from the spirit and scope of the invention as defined by the appended claims. The disclosed embodiments should be considered in descriptive sense only and not for purposes of limitation. Therefore, the scope of the invention is defined not by the detailed description of the invention but by the appended claims, and all differences within the scope will be construed as being included in the present invention.

1. An oral pharmaceutical formulation comprising sustained-release granules containing tamsulosin hydrochloride,
wherein the sustained-release granules comprises about 10 parts to about 300 parts by weight of polyvinyl acetate, about 5 parts to about 250 parts by weight of hydroxypropyl methylcellulose, and about 1 part to about 450 parts by weight of a diluting agent with respect to 1 part by weight of tamsulosin hydrochloride, and
a weight ratio of the sustained-release granules with respect to 1 part by weight of tamsulosin hydrochloride is about 360 to 495 parts by weight.

2. The oral pharmaceutical formulation of claim 1, wherein the sustained-release granules comprises, with respect to 1 part by weight of tamsulosin hydrochloride, about 25 parts to about 150 parts by weight of polyvinyl acetate, about 5 parts to about 100 parts by weight of hydroxypropyl methylcellulose, and about 1 part to about 400 parts by weight of the diluting agent.

3. The oral pharmaceutical formulation of claim 1, wherein the hydroxypropyl methylcellulose has a viscosity of about 10,000 to about 100,000 cPs.

4. The oral pharmaceutical formulation of claim 1, wherein the diluting agent is selected from the group consisting of lactose, microcrystalline cellulose, dibasic calcium phosphate, dibasic calcium phosphate dihydrate, tribasic calcium phosphate, and any combinations thereof.

5. The oral pharmaceutical formulation of claim 1, wherein the sustained-release granules are additionally coated with a sustained-release coating material.

6. The oral pharmaceutical formulation of claim 5, wherein the sustained-release coating material is a polymeric coating material or an enteric coating material.

7. The oral pharmaceutical formulation of claim 1, wherein the sustained-release granules has a sphericity of about 0.85 or greater.

8. The oral pharmaceutical formulation of claim 1, wherein the pharmaceutical formulation is in the form of a capsule comprising the sustained-release granules.

9. The oral pharmaceutical formulation of claim 1, wherein, as evaluated using Dissolution method II (paddle method) in the U.S. Pharmacopoeia (USP) including a dissolution test in 500 mL of a pH 1.2 aqueous buffer solution at about 37±0.5° C. at about 100 rpm for about 2 hours, continuously followed by a dissolution test in a pH 7.2 aqueous buffer solution at about 37±0.5° C. at about 100 rpm for about 8 hours, the oral pharmaceutical formulation has a dissolution rate of the tamsulosin hydrochloride of less than about 40 wt % as a result of the dissolution test for about 2 hours in the pH 1.2 aqueous buffer solution, and a dissolution rate of the tamsulosin hydrochloride of about 80 wt % or greater as a result of the dissolution test for about 8 hours in the pH 7.2 aqueous buffer solution.

10. The oral pharmaceutical formulation of claim 1, wherein the amount of the tamsulosin hydrochloride is about 0.2 mg to about 0.8 mg per single unit dosage form.

11. The oral pharmaceutical formulation of claim 8, wherein the capsule is a capsule of No. 3 and comprises about 0.4 mg or more of the tamsulosin hydrochloride per single unit dosage form.

12. The oral pharmaceutical formulation of claim 1, wherein the oral pharmaceutical formulation is for treatment of benign prostatic hypertrophy.

13. A method of preparing an oral pharmaceutical formulation of claim 1, the method comprising:
mixing and grinding a mixture of about 10 parts to about 300 parts by weight of polyvinyl acetate, about 5 parts to about 250 parts by weight of hydroxypropyl methylcellulose, and about 1 part to about 450 parts by weight of a diluting agent with respect to 1 part by weight of tamsulosin hydrochloride to form granules; and
spheronizing the formed granules with a spheronizer at a rotation speed of about 600 rpm to about 800 rpm for about 15 to about 35 minutes to obtain spheronized sustained-release granules.

14. The method of claim 13, further comprising filling a capsule with the spheronized sustained-release granules to form a capsule formulation.
